(19) United States
(12) Patent Application Publication
(10) Pub. No.: US 2011/0201971 A1
(43) Pub. Date: Aug. 18, 2011

(54) LAUGHTER MEASUREMENT METHOD AND APPARATUS

(76) Inventors: Yohji Kimura, Kyoto (JP); Kazue Kimura, Kyoto (JP)

(21) Appl. No.: 12/735,778

(22) PCT Filed: Jul. 4, 2008
(86) PCT No.: PCT/JP2008/062161

§ 371 Date: Oct. 14, 2010

(30) Foreign Application Priority Data
Feb. 19, 2008 (JP) 2008-038078

(51) Int. Cl. A61B 5/11

(52) U.S. Cl. 600/595

(57) ABSTRACT

A concrete means for accurately detecting laughter such as “suppressed laughter” which is hard to appear on an expression for quantifying. Provided is a concrete means for measuring changes with time of skin surface potential in the vicinity of a xyphoid of a subject or the vicinity of a region on a seventh costa located approximately 10 cm right from the xyphoid in an approximately horizontal direction and calculating changes with time of strength of each frequency of potential measurement waves which have been measured. In addition, the calculated data is mapped so that the changes with time of the strength of each frequency can be identified by color or the like, while one axis is specified to be a frequency axis and the other is a time axis. A concrete means for detecting the laughter of the subject by comparing with a prepared laughter reference pattern is provided.

### TECHNICAL FIELD

The present invention relates to a measurement method and a measurement apparatus that identify laughter of a subject and that quantify the magnitude of the laughter.

### BACKGROUND ART

Medical effects caused by laughter have recently drawn attention and the effects such as improvement of the immunological function due to laughter, etc., have been reported. While the excellent effects caused by laughter have been reported as above, development, etc., of techniques of identifying and quantifying laughter are further executed to scientifically execute researches on laughter and its medical effects and to supply an amount of laughter in the daily life to users as useful information. For example, the following inventions have been made as such techniques.

According to the invention of Patent Document 1, vibration data of the abdomen of a subject and sound data generated by the subject are collected and laughter of the subject is measured and quantified from these pieces of data.

According to the invention of Patent Document 2, sound data is collected from the throat of a subject and laughter of the subject is measured and quantified from the data. Patent Document 1: Japanese Laid-Open Patent Publication No. 2003-319926 Patent Document 2: Japanese Laid-Open Patent Publication No. 2004-243023

### DISCLOSURE OF THE INVENTION

Problems to be Solved by the Invention

However, a direct physical movement caused by laughter is “a sudden and fierce vibration of a diaphragm” and vibrations of an abdomen (abdominal transverse muscle) and a throat (throat muscle group) are only side movements. Therefore, non-genuine laughter such as a “vacuous laugh” or an “ingratiating smile” is also measured according to the inventions of Patent Documents 1 and 2 according to which laughter is measured and quantified based on the data on the vibrations of the abdomen and the throat . In addition, laughter that tends not to be apparent such as “hushed laughter” can not sufficiently be measured. Therefore, the precision of the measurement is not fully satisfactory.

Therefore, an object of the present invention is to provide a specific means for quantifying the genuine laughter caused by “funniness” excluding the non-genuine laughter such as a “vacuous laugh” or an “ingratiating smile”. Another object of the present invention is to provide a specific means for accurately detecting and quantifying laughter including laughter that tends not to be apparent such as “hushed laughter”.

### MEANS FOR SOLVING THE PROBLEM

The following inventions, etc., are provided as the means for solving the problems.

Genuine laughter caused by “funniness” is primarily projected onto a diaphragm. The present invention provides a means for qualitatively and quantitatively analyzing the aspect of a vibration of the diaphragm by measuring a skin surface potential in the vicinity of the starting portion of the diaphragm (that is, xiphoid process, the seventh to the twelfth ribs, and the lumber vertebra) over time and calculating an amount of variation of the potential in a short time for each frequency.

More specifically, the following inventions are provided.

A first invention provides a laughter measurement method comprising a potential measuring step for measuring a skin surface potential of a surface of a bone tissue that is coupled with a tendon of a starting portion of a diaphragm or the starting portion of the diaphragm of a subject over time; and a calculating step for calculating a variation over time of an intensity of each frequency of a measurement wave that represents the variation over time of the potential measured.

A second invention provides the laughter measurement method based on the first invention, wherein the surface of the bone tissue that is coupled with the tendon of the starting portion of the diaphragm or the starting portion of the diaphragm of the subject is vicinity of xiphoid process.

A third invention provides the laughter measurement method based on the first invention, wherein the surface of the bone tissue that is coupled with the tendon of the starting portion of the diaphragm or the starting portion of the diaphragm of the subject is vicinity of a location on a seventh rib that is positioned about 10 cm away and substantially horizontally rightward from the xiphoid process.

A fourth invention provides the laughter measurement method based on anyone of the first to third inventions, further comprising a preparing step for preparing a reference pattern formed by mapping an intensity pattern of each frequency using one axis as a frequency axis and another axis as a time axis, as a reference pattern of a measurement wave measured during laughter; a mapping step for sampling intensity data of each frequency in the measurement wave of the subject calculated at the calculating step and mapping the intensity data sampled of the subject using one axis as a frequency axis and another axis as a time axis; a comparing step for comparing the intensity pattern of the subject mapped at the mapping step with the reference pattern prepared in advance at the preparing step; and a comparison result output step for outputting information that identifies whether laughter is detected according to a result of the comparison at the comparing step.

A fifth invention provides the laughter measurement method based on any one of the first to fourth inventions, further comprising a laughter amount output step for calculating and outputting an index that indicates magnitude of laughter according to a result of the calculation at the calculating step.

A sixth invention provides a laughter measurement apparatus comprising a potential measuring portion that measures a skin surface potential of a surface of a bone tissue that is coupled with a tendon of a starting portion of a diaphragm or the starting portion of the diaphragm of a subject over time; and a calculating portion that calculates a variation over time of an intensity of each frequency of a measurement wave that represent the variation over time of the potential measured.

A seventh invention provides the laughter measurement apparatus based on the sixth invention, wherein the potential measuring portion comprises a xiphoid process vicinity measuring means that measures a skin surface potential in the vicinity of the xiphoid process over time as the skin surface potential of the surface of the bone tissue that is coupled with the tendon of the starting portion of the diaphragm or the starting portion of the diaphragm of the subject.

An eighth invention provides the laughter measurement apparatus based on the sixth invention, wherein the potential measuring portion comprises a seventh rib vicinity measuring means that measures a skin surface potential in the vicinity of a location on a seventh rib that is positioned about 10 cm away and substantially horizontally rightward from the xiphoid process over time as the skin surface potential of the surface of the bone tissue that is coupled with the tendon of the starting portion of the diaphragm or the starting portion of the diaphragm of the subject.

A ninth invention provides the laughter measurement apparatus based on any one of the sixth to eighth inventions, wherein the potential measuring portion is installed in a housing, and wherein an electrode to measure a potential by contacting with a skin of a human is provided on one side of the housing.

A tenth invention provides the laughter measurement apparatus based on any one of the sixth to ninth inventions, further comprising a reference pattern retaining portion that retains a reference pattern formed by mapping an intensity pattern of each frequency using one axis as a frequency axis and another axis as a time axis, as a reference pattern of a measurement wave measured during laughter; a mapping portion that samples intensity data of each frequency in the measurement wave of the subject calculated by the calculating portion, the mapping portion mapping the intensity data sampled of the subject using one axis as a frequency axis and another axis as a time axis; a comparing portion that compares the intensity pattern of the subject mapped by the mapping portion with the reference pattern retained in the reference pattern retaining portion; and a comparison result outputting portion that outputs information that identifies whether laughter is detected according to a result of the comparison by the comparing portion.

An eleventh invention provides the laughter measurement apparatus based on any one of the sixth to tenth inventions, further comprising a laughter amount outputting portion that calculates and outputs an index that indicates magnitude of laughter according to a result of the calculation by the calculating portion.

### EFFECT OF THE INVENTION

According to the laughter measurement method and the apparatus therefor of the present invention, only genuine laughter caused by “funniness” can be detected and quantified excluding a “vacuous laugh” or an “ingratiating smile”. Laughter that tends not to be apparent such as “hushed laughter” can also accurately be detected. The magnitude of the laughter detected can be quantified to be usable as comparative data. As a result, scientific researches are ensured that are executed on the medical effects achieved by genuine laughter on the mind and the body.

Because the detected magnitude of laughter can be quantified to be comparable to the magnitude of a different laughter, the apparatus is usable as a tool to check the health of a subject and the apparatus can also provide objective evaluation (result of examination) in a comical performance contest, etc.

### BRIEF DESCRIPTION OF DRAWINGS

FIG. 1 is a diagram of a relation among xiphoid process, a diaphragm, and the vicinity of a location on a seventh rib.

FIG. 2 is a functional block diagram of a laughter measurement apparatus of a first embodiment.

FIG. 3 is a diagram of the state where potential measuring portions are attached to the vicinity of xiphoid process.

FIG. 4 is a diagram of an example of a potential measurement wave measured by the potential measuring portion.

FIG. 5 is an exemplary diagram of the state where the potential measurement wave is frequency-analyzed.

FIG. 6 is a functional block diagram of a laughter measurement apparatus of a second embodiment.

FIG. 7a is a diagram of an example of a reference pattern of a bursting-out laughter reaction (adult).

FIG. 7b is a diagram of an example of measurement data of the bursting-out laughter reaction (adult).

FIG. 7c is a diagram of an example of measurement data of a surprise laughter reaction (adult).

FIG. 7d is a diagram of an example of measurement data of a big laughter reaction (adult).

FIG. 7e is a diagram of an example of measurement data of a crying reaction (baby).

FIG. 7f is a diagram of an example of measurement data of a sneezing reaction (adult).

FIG. 7g is a diagram of an example of measurement data of a cough reaction (adult).

FIG. 7h is a diagram of an example of measurement data of a heart beat reaction (adult).

FIG. 8 is a diagram of an example of the hardware configuration of the second embodiment.

FIG. 9 is a flowchart of a flow of processes of the second embodiment.

FIG. 10 is a functional block diagram of a laughter measurement apparatus of a third embodiment.

FIG. 11a is a first diagram of an example of a housing accommodating therein the potential measuring portion.

FIG. 11b is a second diagram of the example of the housing accommodating therein the potential measuring portion.

FIG. 11c is a third diagram of the example of the housing accommodating therein the potential measuring portion.

FIG. 12a is a diagram of a first exemplary output of a laughter amount calculated.

FIG. 12b is a diagram of a second exemplary output of the laughter amount calculated.

FIG. 13 is a diagram of the state of a subject during three-point measurement.

FIG. 14a is a diagram of an example of a potential measurement wave of the big laughter reaction (adult).

FIG. 14b is a diagram of an example of a potential measurement wave of a hushed laughter 1 (a suppressed laugh, a silent laugh) reaction (adult).

FIG. 14c is a diagram of an example of a potential measurement wave of a hushed laughter 2 (a snorting laugh, bursting-out laughter) (adult).

FIG. 14d is a diagram of an example of a potential measurement wave of an ingratiating smile reaction (adult).

FIG. 14e is a diagram of an example of a potential measurement wave of a non-laughter state (ordinary state) (adult).

FIG. 15 is a diagram of a result of three-point measurement.

### EXPLANATIONS OF REFERENCE NUMERALS

0601 potential measuring portion

0602 calculating portion

0603 reference pattern retaining portion

0604 mapping portion

0605 comparing portion

0606 comparison result output portion

### PREFERRED EMBODIMENTS OF THE INVENTION

The best embodiments for carrying out the present invention will be described in detail with reference to the accompanying drawings. The present invention is not at all limited to the embodiments and can be carried out in various aspects within the scope thereof not departing from the gist thereof.

The relations between the embodiments and claims are as follows. In a first embodiment, claims 1, 2, 3, 6, 7, 8, etc., are mainly described. In a second embodiment, claims 4, 10, etc., are mainly described. In a third embodiment, claims 5, 11, etc., are mainly described. Ina fourth embodiment, claims 9, etc., are mainly described.

### <<First Embodiment>>

### <Overview of First Embodiment>

A laughter measurement method and a laughter measurement apparatus of a first embodiment are characterized in that the method and the apparatus can accurately detect and quantify data on a vibration movement of a diaphragm. A specific means measures the variation over time of a skin surface potential of a surface of a bone tissue that is coupled with a tendon of the starting portion of the diaphragm or the starting portion of the diaphragm (examples: a skin surface potential of each of “the vicinity of xiphoid process”, “the vicinity of a location on the seventh rib that is positioned about 10 cm away and substantially horizontally rightward from the xiphoid process”, etc., of a subject) as data on a vibration movement of the diaphragm. The method and the apparatus of the embodiment are also characterized in that the method and the apparatus calculate the variation over time of the intensity of each frequency of a potential measurement wave using integral calculus, etc.

### <Functional Configuration of First Embodiment>

FIG. 2 depicts an example of functional blocks of the laughter measurement apparatus of the embodiment. As depicted in FIG. 2, the laughter measurement apparatus of the embodiment includes a “potential measuring portion” (0201) and a “calculating portion” (0202). The “potential measuring portion” (0201) may include either one or more of a “xiphoid-process-vicinity measuring means” and a “seventh-rib-vicinity measuring means”.

The laughter measurement method of the embodiment includes a “potential measurement step” and a “calculation step”. The laughter measurement method of the embodiment can be realized using the laughter measurement apparatus of the embodiment, etc. Details of the laughter measurement apparatus will be described below.

The functional blocks of the apparatus can be realized as hardware, software, or both of the hardware and the software. More specifically, when the apparatus uses a computer, the functional blocks can be: hardware components such as a CPU, a RAM, a bus or a secondary storing apparatus (a storage medium such as a hard disc, a non-volatile memory, a CD-ROM, and a DVD-ROM, and a reading drive for these media, etc.), a printing device, a displaying apparatus, and other external peripheral apparatuses, etc.; and an I/O port for the external peripheral devices, a driver program to control the hardware components, other application programs, a user interface used to input information, etc.

These hardware and software components are used to: compute and process programs read into the RAM using the CPU; to process, accumulate, and output-process data stored in the memory or on the hard disc and data input through the interface, etc.; or to control the hardware components, etc. The present invention can not only be realized as an apparatus but also be realized as a method. A portion of the present invention can be configured as software. A software product used to cause a computer to execute such software, and a storage medium formed by fixing the product on a storage medium are naturally encompassed in the technical scope of the present invention (throughout the whole specification).

The “potential measuring portion” (0201) is adapted to measure a skin surface potential of the surface of a bone tissue that is coupled with a tendon of the starting portion of the diaphragm or the starting portion of the diaphragm of a subject over time. The “starting portion of the diaphragm” refers to the starting portion of the diaphragm and it is known that the starting portion of the diaphragm is couple with the “xiphoid process of the sternum”, the “inner faces of the seventh to the twelfth costal cartilages (costal arch)”, and “the first to the third lumber bodies”. The potential measuring portion (0201) is adapted to measure the skin surface potential in the vicinity of these (“xiphoid process of the sternum”, the “inner faces of the seventh to the twelfth costal cartilages (costal arch)”, and “the first to the third lumber bodies”).

The “xiphoid-process-vicinity measuring means” of the “potential measuring portion” (0201) is adapted to measure a skin surface potential in the vicinity of the xiphoid process over time as the skin surface potential of the surface of the bone tissue that is coupled with the tendon of the starting portion of the diaphragm or the starting portion of the diaphragm of the subject. The “seventh-rib-vicinity measuring means” of the “potential measuring portion” (0202) is adapted to measure a skin surface potential in the vicinity of a location on the seventh rib that is positioned about 10 cm away and substantially horizontally rightward from the xiphoid process (hereinafter, “vicinity on the seventh right rib”) over time as the skin surface potential of the surface of the bone tissue that is coupled with the tendon of the starting portion of the diaphragm or the starting portion of the diaphragm of the subject.

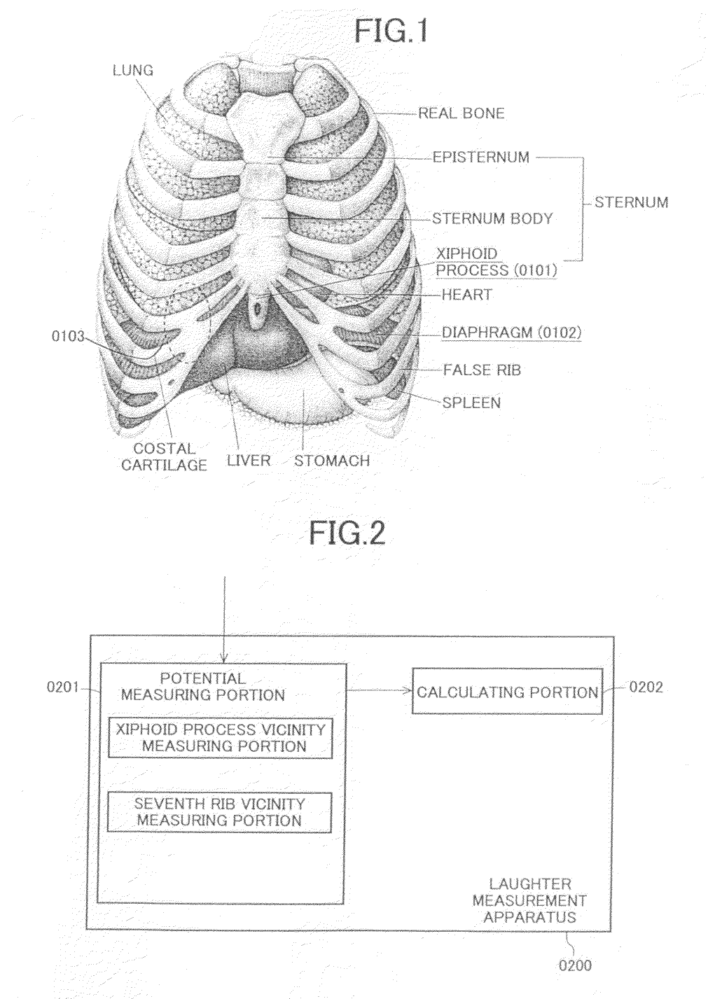

As depicted in FIG. 1, the “xiphoid process” (0101) is a kind of sternum that is present around the pit of the stomach and, as above, is coupled with the starting portion of the diaphragm (0102). Therefore, a vibration movement of the diaphragm can be accurately detected by measuring the skin surface potential in the vicinity of the xiphoid process. In FIG. 1, the “vicinity on the seventh right rib” is the vicinity indicated by a dotted circle (0103) and, as above, the diaphragm adheres to the tendon in the vicinity of the seventh right rib. Therefore, the vibration movement of the diaphragm can accurately be detected by measuring the skin surface potential in the vicinity on the seventh right rib.

The purpose that the “potential measuring portion” (0201) measures the skin surface potential on the surface of the bone tissue that is coupled with the tendon of the starting portion of the diaphragm or the starting portion of the diaphragm of the subject over time is to accurately detect the vibration movement of the diaphragm. Therefore, the location to measure the skin surface potential is not limited to the surface of the bone tissue that is coupled with the tendon of the starting portion of the diaphragm or the starting portion of the diaphragm of the subject when the vibration movement of the diaphragm can accurately be detected at other locations. For example, a skin surface potential may be measured of a neck through which the phrenic nerve that controls the diaphragm runs, etc. The same precondition is applied for all the embodiments below.

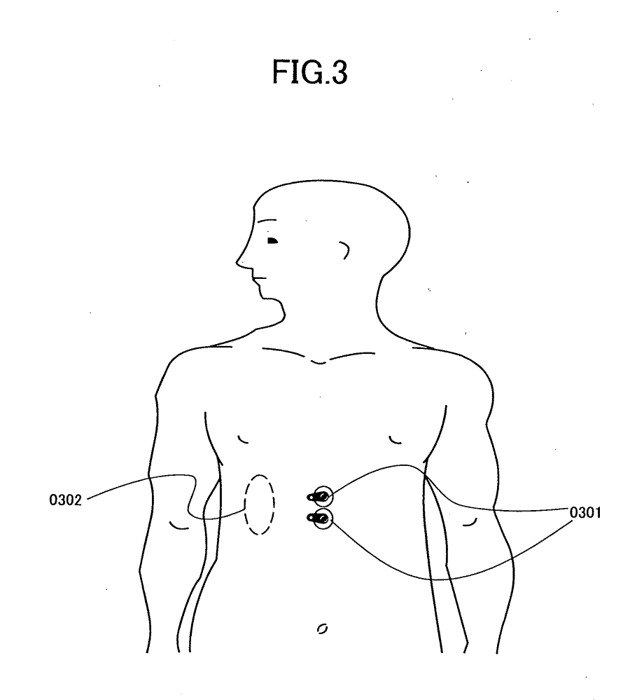

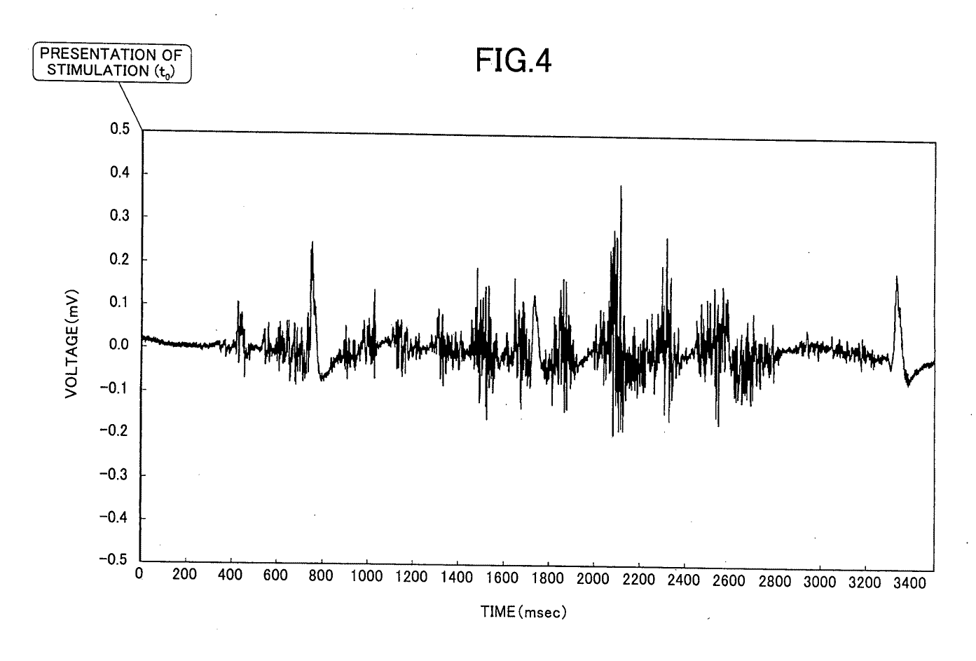

For example, a means that is the same as that of a surface electromyograph examination that is a conventional technique can be used as the means for measuring the variation over time of the skin surface potential on the surface of the bone tissue that is coupled with the tendon of the starting portion of the diaphragm or the starting portion of the diaphragm of the subject (such as the “vicinity of the xiphoid process” and the “vicinity on the seventh right rib”). More specifically, two electrodes are attached, at an interval of about 3 cm, to the skin surface of the surface of the bone tissue that is coupled with the tendon of the starting portion of the diaphragm or the starting portion of the diaphragm of the subject (such as the “vicinity of the xiphoid process” and the “vicinity on the seventh right rib”) and, thereby, the muscle action potential is measured. The reason why the two electrodes are attached is because the difference in the voltage between the two electrodes is measured. The apparatus can be realized with the conditions for the measurement, etc., that are the same as the conventional technique (the surface electromyograph examination). For example, the measurement is enabled with the measurement sensitivity of about several μV to several 10 mV. The precision of the measurement may be precision of collecting data at a rate of 3,000 times/second. (This value may be varied according to the purpose of use of the laughter measurement apparatus. The same precondition as above is applied to all the embodiments below.) The xiphoid process can easily be found by touching with a finger from the surface of the skin. Starting from this point, the vicinity on the seventh right rib can also easily be found. FIG. 3 depicts the state where the two electrodes (0301) are attached to the skin surface in the xiphoid process vicinity of the subject. The vicinity on the seventh right rib is indicated by a dotted circle (0302). FIG. 4 depicts an example of a potential measurement wave measured by the potential measuring portion (0201). In FIG. 4, the axis of abscissa represents time and the axis of ordinate represents voltage.

The “calculating portion” (0202) is adapted to calculate the intensity of each frequency of a measurement wave and its variation over time that is the variation over time of the potential measured. The “measurement wave that is the variation over time of the measured potential”: is a potential measurement wave that is measured by the potential measuring portion (0201) for a specific time period from the skin surface of the surface of the bone tissue that is coupled with the tendon of the starting portion of the diaphragm or the starting portion of the diaphragm of the subject (such as the “vicinity of the xiphoid process” and the “vicinity on the seventh right rib”); and is a waveform depicted in FIG. 4, etc. A specific means for calculating “the intensity of each frequency of a measurement wave and its variation over time” frequency-analyzes the potential measurement wave obtained using wavelet transformation or short-time Fourier transformation, etc. first. “The intensity of each frequency and its variation over time” can be obtained by integral calculus for each frequency. The data obtained can be used in inventions, etc., described in a second embodiment below.

By applying an adding processing to the result, “the total amount of intensity of all the frequencies and its variation over time (the variation over time of the intensity of the measurement wave)” may be obtained. The potential measurement wave is caused by the vibration movement of the diaphragm measured from the skin surface of the surface of the bone tissue that is coupled with the tendon of the starting portion of the diaphragm or the starting portion of the diaphragm (such as the “xiphoid process” and the “vicinity on the seventh right rib”) of the subject. “The variation over time of the total amount of the intensity of all the frequencies (the variation over time of the intensity of the measurement wave)” is obtained by quantifying the variation over time of the magnitude of the vibration movement of the diaphragm.

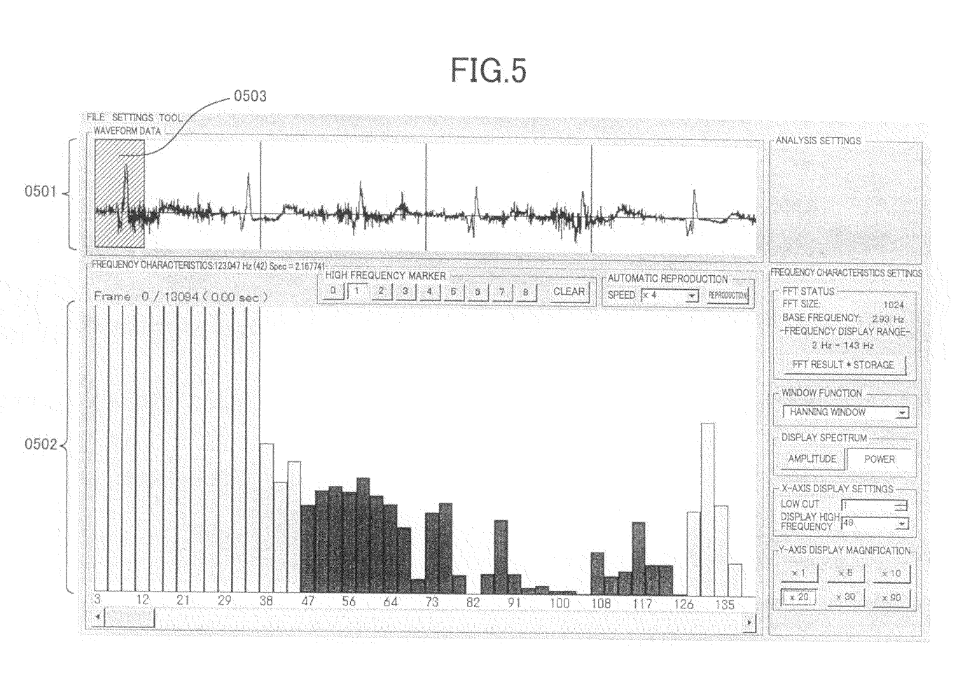

FIG. 5 depicts an example of a screen displaying the state where the potential measurement wave is frequency-analyzed. The upper portion of the screen (0501) shows a waveform of the potential measurement wave that is measured from the skin surface of the surface of the bone tissue that is coupled with the tendon of the starting portion of the diaphragm or the starting portion of the diaphragm (such as “the vicinity of xiphoid process” and “the vicinity on the seventh right rib”) of the subject. In the upper portion of the screen, the axis of abscissa represents time and the axis of ordinate represents voltage. The lower portion of the screen (0502) shows a graph represents the result of execution of the frequency-analysis and the integral calculus for the potential measurement wave of a temporal region identified by the shadowed portion (0503) in the potential measurement wave in the upper portion of the screen (0501). In the lower portion of the screen, the axis of abscissa represents frequency and the axis of ordinate represents intensity. The screen is an example of a screen that displays the result of the computation, and the laughter measurement method and the apparatus therefor of the embodiment are not limited to the above.

In FIG. 2, the “potential measuring portion” (0201) and the “calculating portion” (0202) are described to be present in one apparatus. This is because a form is assumed where the “potential measuring portion” (0201) configured by the electrodes attached to the subject, etc., is always connected to the main body of the laughter measurement apparatus that includes the “calculating portion” (0202) with a cord, etc., and the potential measurement wave obtained from the subject is sent to the “calculating portion” (0202) that is present in the main body of the laughter measurement apparatus in real time through the cord, etc.

However, the laughter measurement apparatus of the embodiment is not limited to the form depicted in FIG. 2 and, for example, the “potential measuring portion” (0201) and the “calculating portion” (0202) may not be adapted to be always connected to each other with a cord, etc., and may be adapted to be in the state where the portions are separated from each other as different apparatuses. One laughter measurement apparatus may be configured by using the separate apparatuses as a combination. In this case, the “potential measuring portion” (0201) may also be adapted to be able to have stored therein the potential measurement wave obtained from the subject. The potential measurement wave stored may be adapted to be sent to an apparatus that configures the “calculating portion” using USB communication, infrared communication, Bluetooth communication, etc. The communication may be executed in either of real time processing and batch processing. The details of such a configuration will be described in the fourth embodiment below.

In the above, obtaining the data on the vibration movement of the diaphragm is realized by measuring the potential wave of the skin surface of the surface of the bone tissue that is coupled with a tendon of the starting portion of the diaphragm or the starting portion of the diaphragm (such as “the vicinity of xiphoid process” and “the vicinity on the seventh right rib” of the subject. In addition, the data on the vibration movement of the diaphragm may be obtained by measuring an impulse of the phrenic nerve. The data on the vibration movement of the diaphragm may also be obtained using an electromagnetic or a sonic apparatus such as that using an X ray, echo, or a SUQID.

### <Effects of First Embodiment>

According to the laughter measurement method and the measurement apparatus therefor of the embodiment, the vibration movement of the diaphragm may accurately be detected and the variation over time of its intensity can be calculated as useful comparative data. As a result, various phenomena caused by the vibration movement of the diaphragm can scientifically be studied. More specifically, as described in the embodiments below, laughter can accurately be identified and the magnitude of the laughter can be calculated as the useful comparative data.

### <<Second Embodiment>>

### <Overview of Second Embodiment>

A laughter measurement method and a laughter measurement apparatus of a second embodiment are based on the first embodiment, and further retain reference data on laughter in advance (data on the time period, frequencies, and intensity). After applying predetermined processing such as a computing processing to the potential measuring wave obtained from the subject, the processed data is compared with the reference data. The method and the apparatus of the embodiment are characterized in that laughter is detected from the potential measurement wave obtained from the subject in this manner.

### <Functional Configuration of Second Embodiment>

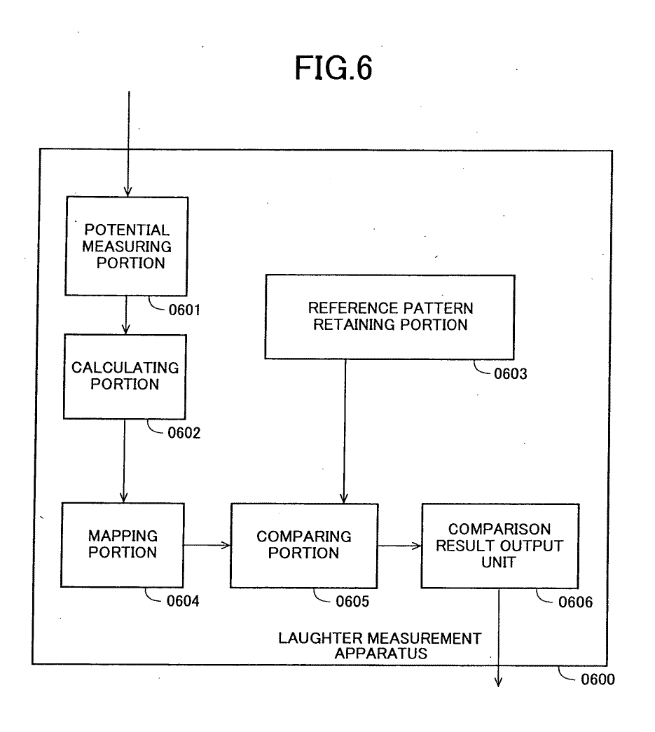

FIG. 6 depicts an example of the functional blocks of the laughter measurement apparatus of the embodiment. As depicted in FIG. 6, the laughter measurement apparatus of the embodiment includes a “potential measuring portion” (0601), a “calculating portion” (0602), a “reference pattern retaining portion” (0603), a “mapping portion” (0604), a “comparing portion” (0605), and a “comparison result output portion” (0606).

The laughter measurement method of the embodiment includes a “potential measuring step”, a “calculating step”, a “preparing step”, a “mapping step”, a “comparing step”, and a “comparison result output step”.

The laughter measurement method of the embodiment can be realized by the laughter measurement apparatus of the embodiment, etc. The details of the laughter measurement apparatus will be described.

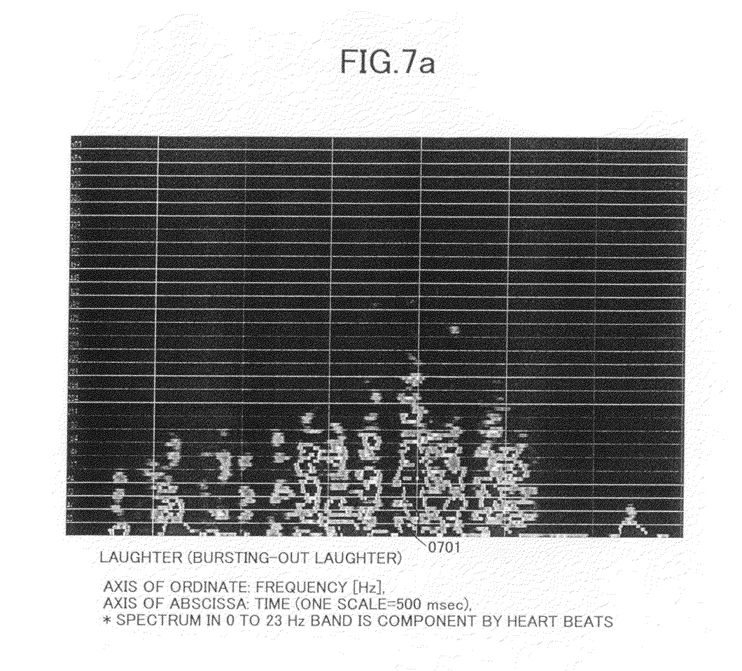

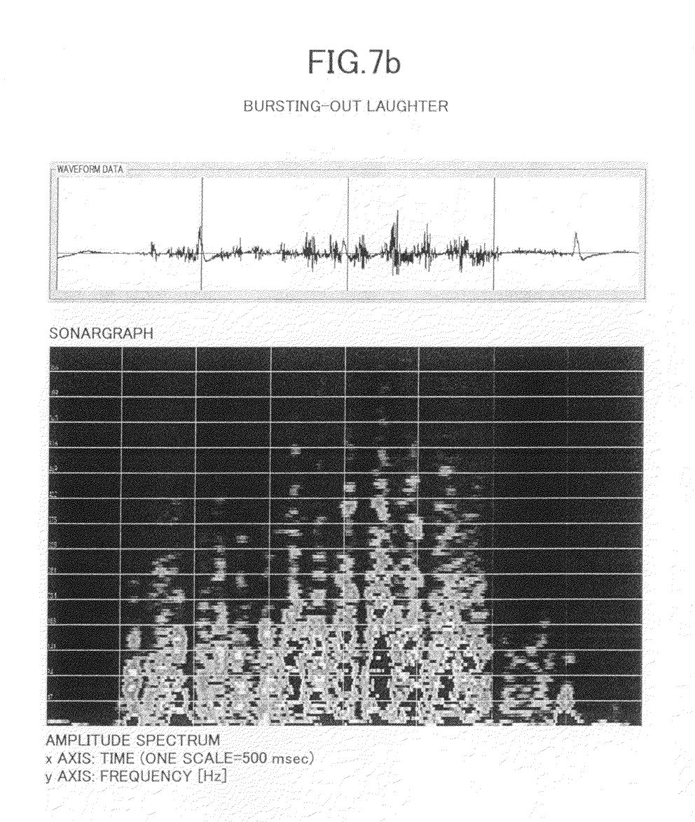

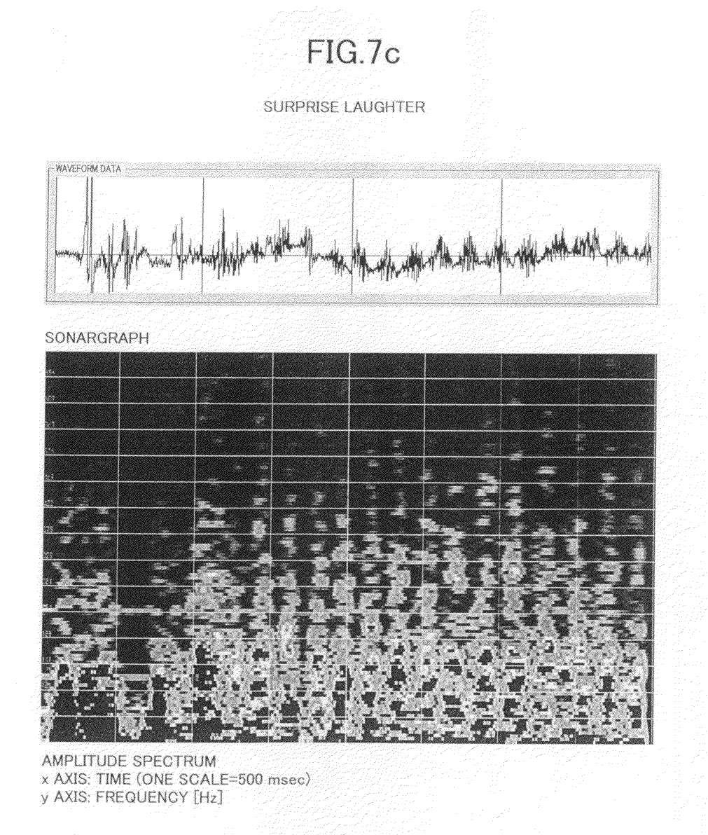

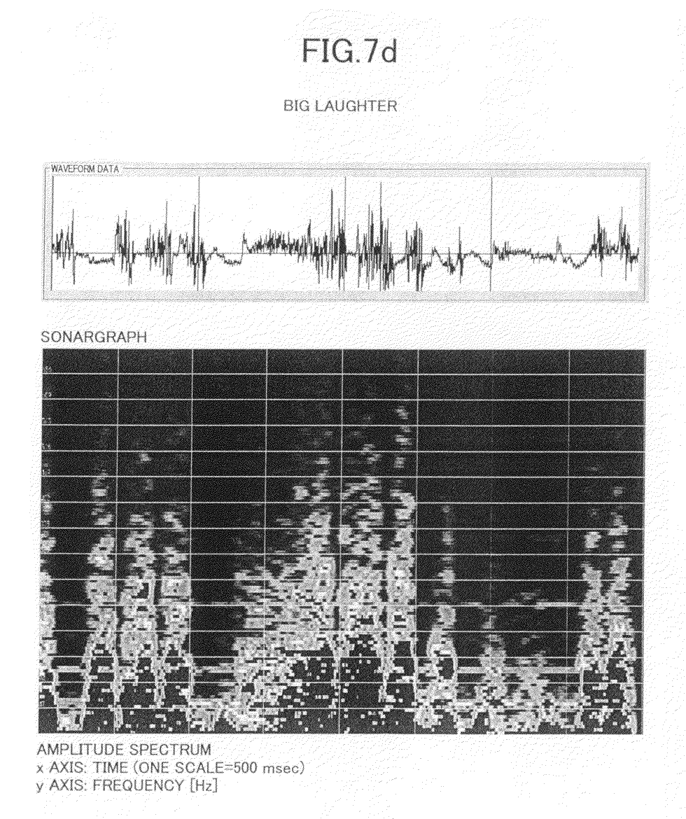

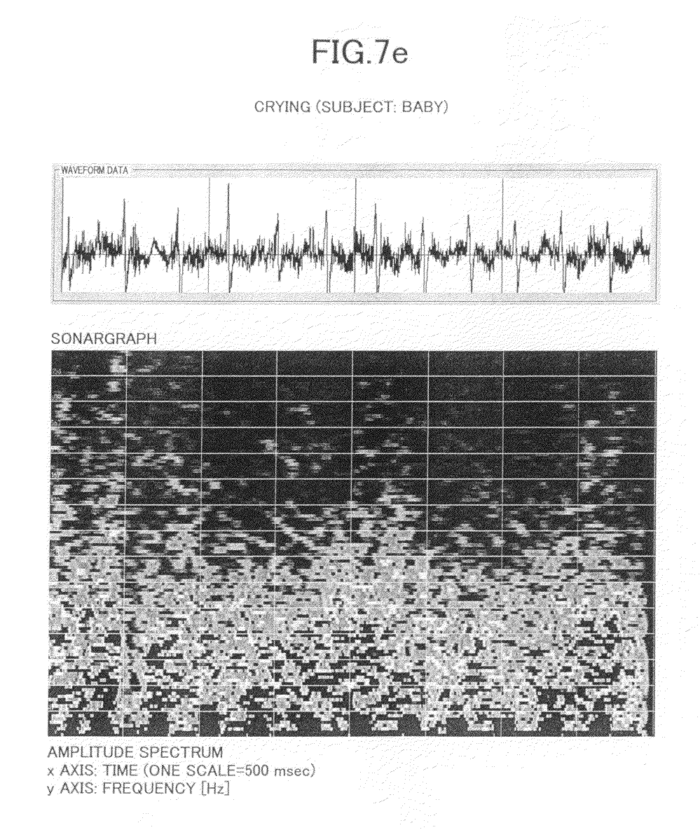

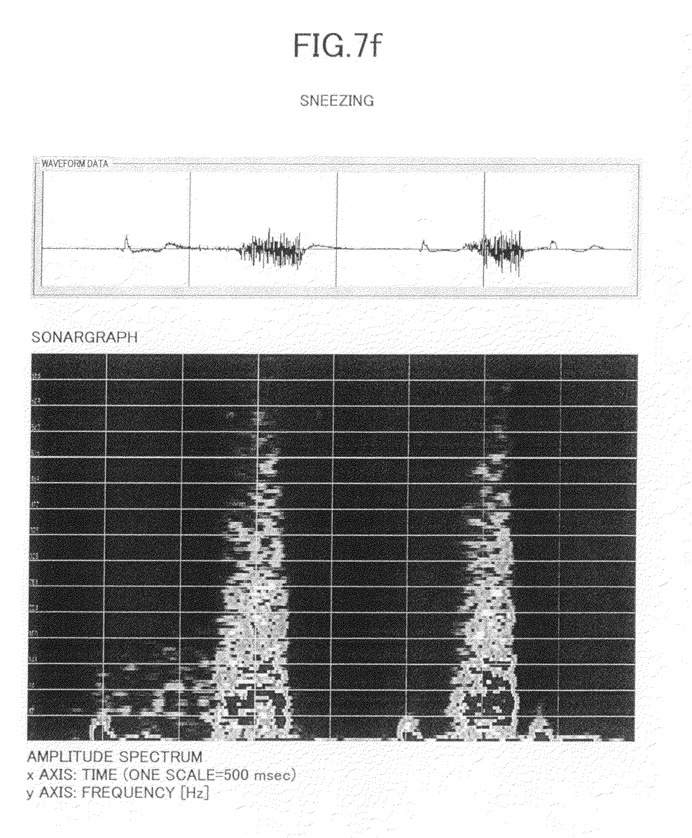

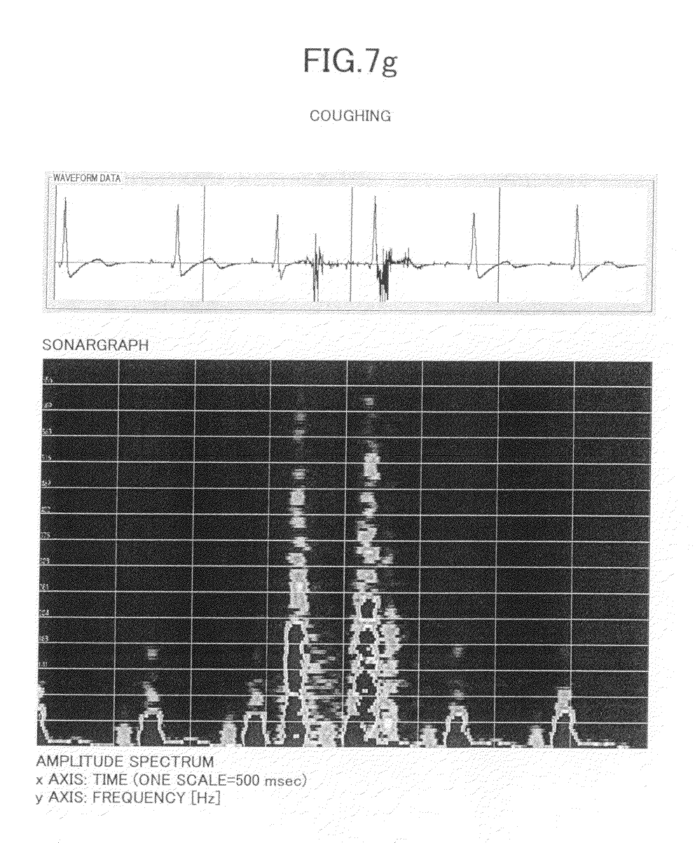

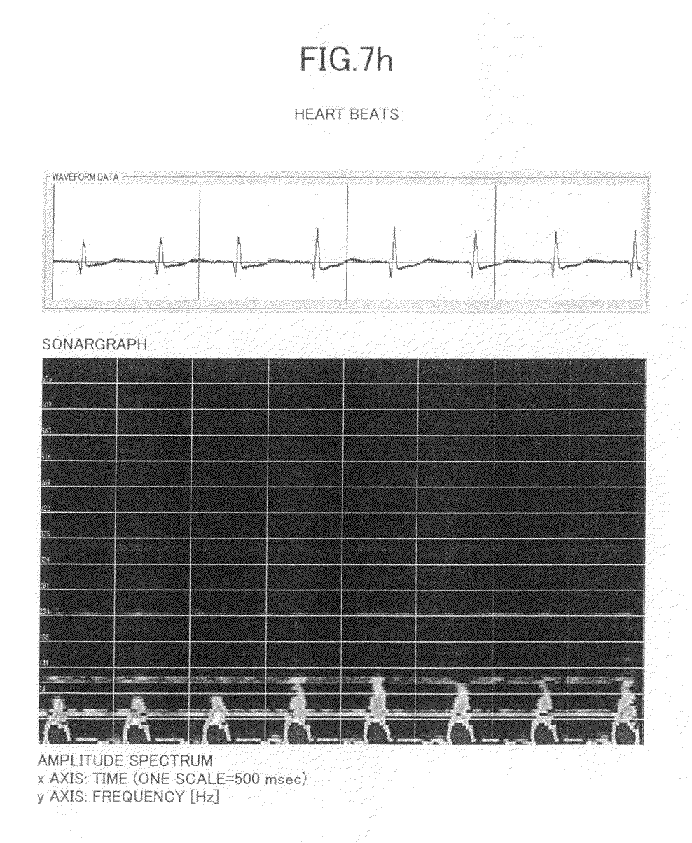

The “reference pattern retaining portion” (0603) is adapted to retain a reference pattern formed by mapping an intensity pattern of each frequency using one axis as a frequency axis and the other axis as a time axis, as a reference pattern of a measurement wave measured during laughter. The “reference pattern” of the measurement wave measured during the laughter is a typical pattern that represents variation over time of the intensity of each frequency of the potential measurement wave measured from the skin surface of a surface of a bone tissue that is coupled with a tendon of the starting portion of the diaphragm or the starting portion of the diaphragm (such as “the vicinity of xiphoid process” and “the vicinity on the seventh right rib”) of the subject when laughter is generated. For example, a determining means thereof may actually measure a large amount of sample data from a large number of persons and may determine the typical pattern using the least square method, etc. For reference, an example of a reference pattern retained by the “reference pattern retaining portion” (0603) is depicted in FIG. 7(a). FIG. 7(a) is a graph of an example of a reference pattern of bursting-out laughter. Using the axis of ordinate as a frequency axis and the axis of abscissa as a time axis, the graph displays the variation over time of the intensity of each frequency that is identifiable using color gradations. More specifically, in FIG. 7(a), the variation over time of the intensity of each frequency is depicted using a plurality of designs longitudinally extending from low frequencies to high frequencies. A thick color (0701) that is present at the innermost position of the design represents the highest intensity and the intensity becomes weaker from the position toward the outside of the design. In FIG. 7(a), the intensity is identifiably displayed using the gradations of the painting. However, the intensity may identifiably be displayed using the kinds of color.

The “reference pattern retaining portion” (0603) may retain only one reference pattern of laughter, or may retain a plurality of reference patterns for each category such as generation (examples: teens, twenties, thirties, etc.), sex (examples: male, female), physical constitution (examples: lean, ordinary, fat, etc.), etc.

The “reference pattern retaining portion” (0603) can realize the retention of reference patterns as above at the “preparing step”. More specifically, at the preparing step, the reference patterns may in advance be stored in the apparatus when the apparatus is shipped. The apparatus may further be adapted to be able to obtain new reference patterns and update those in its retention after the apparatus is shipped as merchandize. The reason why the apparatus is adapted to be able to update is because, as above, the reference patterns may be determined by actually obtaining a large amount of sample data and analyzing the data, and because, in such a case, the number of samples to be obtained is increased as the time elapses and the reference patterns may be varied.

The “mapping portion” (0604) samples intensity data indicating the variation over time of the intensity of each frequency in the potential measurement wave of the subject calculated by the calculating portion (0602) (hereinafter, “subject intensity data”), and is adapted to map the subject intensity data sampled using one axis as a frequency axis and the other axis as a time axis. FIGS. 7(b) to 7(h) depict examples of the subject intensity data mapped by the mapping portion. In each of FIGS. 7(b) to 7(h), “waveform data” in the upper portion depicts an electric measurement wave (the axis of abscissa: time, the axis of ordinate: voltage) measured from the subject, and “sonagraph” in the lower portion depicts a graph of the subject intensity data mapped (the axis of abscissa: time, the axis of ordinate: voltage). FIG. 7(b) depicts the mapped subject intensity data measured during “bursting-out laughter”. FIG. 7(c) depicts that measured during “surprise laughter (laughter after being surprised)”. FIG. 7(d) depicts that measured during “big laughter”. FIG. 7(e) depicts that measured during “crying (subject: a baby)”. FIG. 7(f) depicts that measured during “sneezing”. FIG. 7(g) depicts that measured during “coughing”. FIG. 7(h) depicts that measured in an “ordinary state (data created concerning heart beats)”.

The mapping of the mapping portion (0604) is executed to enable easy grasp of the variation over time of the intensity of each frequency in the measurement wave of the subject. The method of the mapping is the same as that of the reference pattern of FIG. 7(a). (Hereinafter, the subject intensity data mapped is referred to as “subject intensity pattern”.)

The “comparing portion” (0605) is adapted to compare the subject intensity pattern mapped by the mapping portion (0604) with the reference patterns retained in the reference pattern retaining portion (0603). For the comparison, when the reference pattern retaining portion (0603) retains reference patterns of laughter for each of the categories such as generation, only the reference patterns of the category that the subject belongs to may be compared with. “Pattern recognition” maybe used as a specific means of the comparison. Which approach of the pattern recognition is used is not especially limited. However, the inventor of the present invention has found that the potential measurement wave measured when laughter is generated has characteristic points in its frequency band having high intensity and variation thereof over time (the number of times of repetition), etc. Therefore, for the laughter measurement method and the laughter measurement apparatus of the embodiment, it is desired to execute the pattern recognition especially using the characteristic points (the variation overtime of the intensity of each frequency), etc. By doing this, the laughter of the subject can more accurately be detected. The comparison can be executed using as one unit the data for one scale (500 msec) on the axis of abscissa (time axis) depicted in FIG. 7(a). However, preferably, the comparison is executed using the data for about four scales (2 sec) to 12 (6 sec) as one unit to execute the comparison more accurately.

The vibration movement of the diaphragm is also caused by factors other than laughter (examples: “coughing”, “sneezing”, “hiccupping”, etc.) Therefore, to accurately identify laughter, the reference pattern retaining portion (0603) may also be adapted to also retain reference data of the factors other than laughter (examples: “coughing”, “sneezing”, “hiccupping”, etc.) and also compare the subject intensity pattern with the reference data of the factors other than laughter.

The comparison result by the comparing portion (0605) may also be the result that identifies whether laughter is “detected” or “undetected”. When the comparison is executed also using the reference patterns of the factors other than laughter as above, the comparison result may also be the result that identifies the reference pattern that most resembles the subject intensity pattern.

The comparison result can be output for each one unit of the execution of the comparison (examples: “500 msec”, “6 sec”). When the comparison is set to be executed using one scale (500 msec) of the time axis (axis of abscissa) as one unit, the comparison is executed for each one scale (500 msec) and its result is output.

The characteristic points of the reference pattern of laughter will be described with reference to the reference pattern depicted in FIG. 7(a). Differences will be described, between the reference pattern and the pieces of data of the reactions other than the laughter reaction depicted in FIGS. 7(e) to 7(h). As above, the reference pattern may be varied due to further analyses executed in the future. Therefore, those depicted herein are only examples, and the comparing processing by the comparing portion (0605) needs not always to be executed using a pattern that completely coincides with any of the reference patterns described below when the laughter measurement method and the laughter measurement apparatus of the embodiment are executed.

FIG. 7(a) depicts an example of a reference pattern of a laughter reaction (bursting-out laughter, adult). In FIG. 7(a), one scale=500 msec on a time axis (the axis of abscissa). The axis of ordinate is a frequency axis and values are written on the left thereof. Thickly colored lumps each having a longitudinally elongated design that are shown in a third to a fifth scales from the left (1,000 to 2,500 msec) and that are seen around 50 to 200 Hz are the pattern that is specific to laughter. Of the plurality of longitudinally elongated design, each one block-like lump is formed corresponding to generation of laughter that sounds “ha, ha, ha”. The block-like lump that corresponds to laughter appears at a cycle of three to seven times/sec. In FIG. 7(a), thickly colored block-like lumps that are seen around 0 to 50 Hz are caused by the heart beats.

FIG. 7(f) shows a measurement data pattern of a sneezing reaction (adult). In FIG. 7(f), two longitudinally elongated designs are the pattern that is specific to sneezing. Each one block-like lump is formed corresponding to generation of sneezing that sounds “atchoo”. The block-like lump that is specific to the sneezing reaction appears alone or the next block-like lump appears after a relatively long time interval. Therefore, the reactions are not seen at a cycle of three to seven times/sec as seen for the laughter reaction.

FIG. 7(g) shows a measurement data pattern of a coughing reaction (adult). In FIG. 7(g), two longitudinally elongated designs are the pattern that is specific to sneezing. Each one block-like lump is formed corresponding to generation of coughing that sounds “gohon”. The block-like lump that is specific to the coughing reaction appears alone or the next block-like lump appears after a relatively long time interval. Though this time interval is shorter than that of the sneezing reaction, this time interval is longer than that of the laughter reaction. As can be seen from FIG. 7(g), for the coughing reaction, reactions are not seen at a cycle of three to seven times/sec as seen for the laughter reaction.

In the pieces of data shown in FIGS. 7(e) and 7(h) other than that of the laughter reaction, the pattern that is specific to the laughter reaction is apparently not seen.

The “comparison result output portion” (0606) is adapted to output information to identify whether laughter is detected corresponding to the comparison result by the comparing portion. In the case where the result by the comparing portion identifies whether the laughter is “detected” or “undetected”, when the portion (0606) obtains the result that is “detected”, the portion (0606) outputs information that identifies the detection of the laughter, corresponding to the result. In the case where the result by the comparing portion identifies the reference pattern that most resembles the subject intensity pattern, when the portion (0606) obtains the result that represents that the reference pattern identified is “the reference pattern of laughter”, the portion (0606) outputs information that identifies that the laughter is detected, corresponding to the result. The “information that identifies that the laughter is detected” is not especially limited and, for example, may be character information such as “laughter” or “laughter is present” or may be output using pictures (such as a picture of a face, or a picture of the whole body) whose laughter painted therein is identifiable. The comparison result output portion (0606) may also output as the “intensity of laughter” the intensity of the measurement wave (the total amount of the intensity of each frequency in a unit time period) calculated by the calculating portion (0602), together with the information that identifies that the laughter is detected.

The each processing described above and executed by the “calculating portion” (0602), the “mapping portion” (0604), and the “comparing portion” (0605) may be executed removing the data at frequencies of 20 to 50 Hz or lower from the measurement wave measured from the subject. The purpose of this is to remove a vibration movement component of the diaphragm caused by the heart beats. FIG. 7(h) shows the state where any stimulation such as laughter is not at all present, that is, a measurement potential wave (in FIG. 7(h), the upper portion) concerning the vibration movement of the diaphragm caused only by heart beats, and a chart (in FIG. 7(h), the lower portion) obtained by frequency-analyzing the measurement potential wave and mapping the analysis result using one axis as a frequency axis and the other axis as a time axis.

As shown in FIG. 7(h), it can be seen that the vibration movement component of the diaphragm at about 50 Hz or lower caused by the heart beat is detected. The reason why the vibration movement component has a width of 20 to 50 Hz or lower is because the value thereof somewhat differs due to the sex, the physical constitution, age, etc., of each subject. By removing the vibration movement component of the diaphragm caused by the heart beats as above, the magnitude of the laughter can accurately be quantified and, in addition, the comparison with the reference pattern can be executed with higher precision. Naturally, the data at frequencies at 20 to 50 Hz or lower is removed from the reference pattern used in such a case.

### <Hardware Configuration of Second Embodiment>

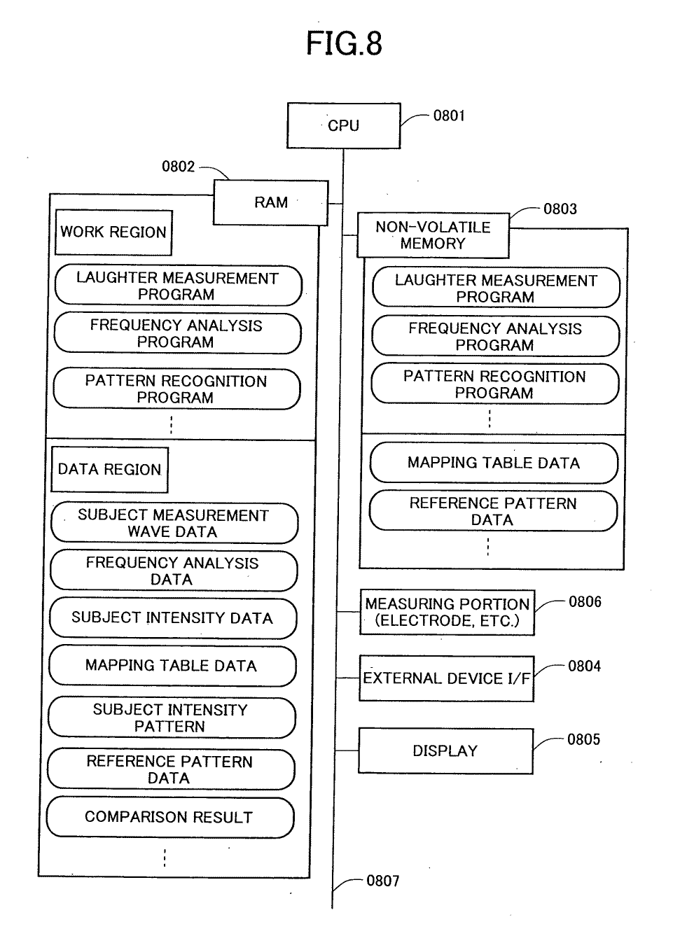

FIG. 8 is a diagram of an example of the configuration employed when the functional configuration is realized as hardware. As depicted in FIG. 8, the portable terminal apparatus includes a “CPU” (0801), a “RAM” (0802), a “non-volatile memory” (0803), an “external device I/F” (0804), a “display” (0805), a “measuring portion” (0806), a “bus” (0807), etc., that configure the “potential measuring portion”, the “calculating portion”, the “reference pattern retaining portion”, the “mapping portion”, the “comparing portion”, the comparison result output portion”, etc.

An example of means for realizing the embodiment will be described with reference to the hardware diagram of FIG. 8.

The CPU (0801) executes a computing processing and executes control processing to control the external device I/F (0804), the display (0805), the measuring portion (0806), etc., according to orders of a laughter measurement program read into the RAM (0802). The CPU (0801) first controls the measuring portion (0806) or the external device I/F (0804) to obtain a subject potential measurement wave according to a subject potential measurement wave obtaining order of the laughter measurement program. The measuring portion (0806) is composed of electrodes that are connected to cords. When the electrodes are attached to the skin surface of the surface of the bone tissue that is coupled with the tendon of the starting portion of the diaphragm or the starting portion of the diaphragm (such as “the vicinity of xiphoid process” and “the vicinity on the seventh right rib”) of the subject, the potential measurement wave can be obtained in real time. On the other hand, the external device I/F (0804) makes USB connection, etc. in advance, with a simple measurement apparatus that has stored therein potential measurement waves obtained from the skin surface of the surface of the bone tissue that is coupled with the tendon of the starting portion of the diaphragm or the starting portion of the diaphragm (such as “the vicinity of xiphoid process” and “the vicinity on the seventh right rib”) of the subject, and, thereby, the I/F (0804) can collectively obtain the stored potential measurement waves. The obtained subject potential measurement wave data is stored in the RAM (0802).

When the CPU (0801) takes out a frequency analysis program according to a frequency analysis order of the laughter measurement program, the CPU (0801) executes a computing processing according to the program and stores its result into the RAM (0802) as frequency analysis data. Thereafter, according to an integration computation order of the laughter measurement program, the CUP (0801) integrates the frequency analysis data stored in the RAM (0802) and stores its result into the RAM (0802) as subject intensity data. When the CPU (0801) takes out mapping table data from the non-volatile memory (0803) according to a mapping order of the laughter measurement program, the CPU (0801) maps the subject intensity data in the mapping table data and stores the mapped data into the RAM (0802) as a subject intensity pattern.

Thereafter, when the CPU (0801) takes the reference pattern data out of the non-volatile memory (0803) according to a comparison order of the laughter measurement program, the CPU (0801) takes out a pattern recognition program and, according to this program, the CPU (0801) compares the subject intensity pattern with the reference pattern and stores the comparison result into the RAM (0802).

When the comparison result is “detection of laughter”, the CPU (0801) takes information that identifies that the laughter is detected out of the non-volatile memory (0803) according to a comparison result output order of the laughter measurement program and controls the display (0805) to display the information.

<Processing flow of Second Embodiment>

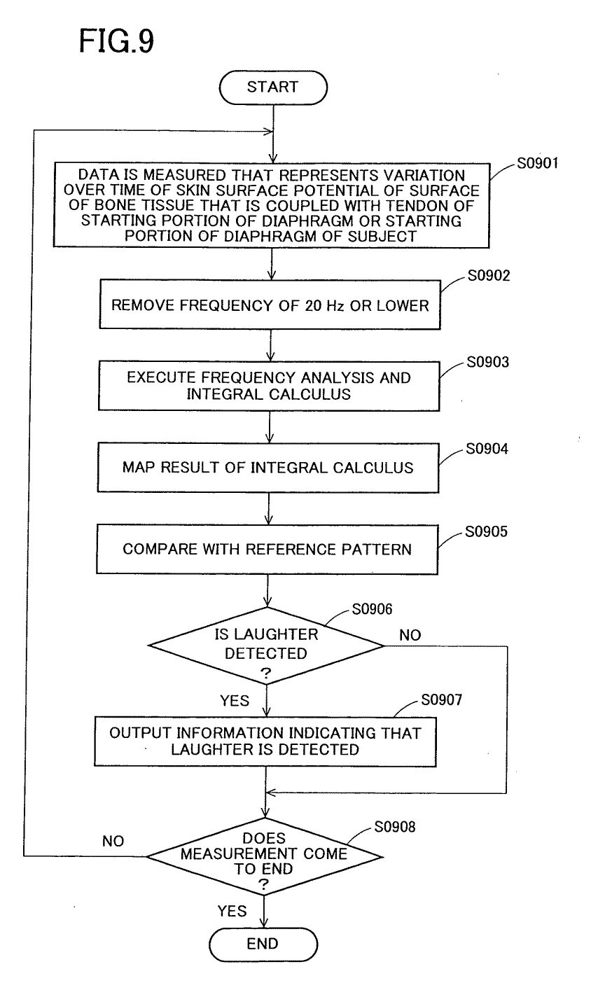

A flowchart of FIG. 9 shows an example of the processing flow of the embodiment. An example of the processing will be described below that are executed when the vibration movement component of the diaphragm caused by the heart beats is determined to be at 20 Hz or lower and this component is removed.

Variation over time is measured of a skin surface potential of the surface of the bone tissue that is coupled with the tendon of the starting portion of the diaphragm or the starting portion of the diaphragm (such as “the vicinity of xiphoid process” and “the vicinity on the seventh right rib”) of the subject (S0901). After removing frequencies lower than 20 Hz from the measured potential measurement wave (S0902), the measured potential measurement wave is frequency-analyzed and its result is integrated (S0903).

Thereafter, the result of the integration is mapped (S0904) and the result of the mapping is compared with the reference pattern (S0905).

When laughter is detected as the result of the comparison (S0906), the information that identifies that laughter is detected is output (S0907). Thereafter, until the measurement comes to an end (S0908), the processes (S0901 to S0907) are repeated.

### <Effects of Second Embodiment>

The laughter measurement method and the measurement apparatus of the embodiment enables accurate recognition of laugher of a subject. The magnitude of the laughter can also be measured.

As a result, influences of laughter and the magnitude of the laughter on a human can scientifically be studied and, in addition, laughter of audiences can accurately be quantified in a comical performance event, etc., and objective information as to whether the performance is funny can also be provided. The amount of laughter of a subject in his/her daily life, etc., can also be grasped.

### <<Third Embodiment>>

### <Overview of Third Embodiment>

A laughter measurement method and a laughter measurement apparatus of a third embodiment are based on those of the first or the second embodiment and are characterized in that the method and the apparatus convert the magnitude of the diaphragm vibration movement calculated by the integration-computation into a form that is understandable for a subject as information indicating the magnitude of the laughter, and provide the information.

### <Functional Configuration of Third Embodiment>

FIG. 10 depicts an example of functional blocks of the laughter measurement apparatus of the embodiment. As depicted in FIG. 10, the laughter measurement apparatus of the embodiment includes a “potential measuring portion” (1001), a “calculating portion” (1002), and a “laughter amount output portion” (1007). The laughter measurement method of the embodiment includes a “potential measuring step”, a “calculating step”, and a “laughter amount output step”. The laughter measurement method of the embodiment can be realized by the laughter measurement apparatus of the embodiment, etc. The details of the laughter measurement apparatus will be described.

The “laughter amount output portion” (1007) is adapted to calculate an index that indicates the magnitude of laughter corresponding to the magnitude of the calculation result by the calculating portion (1002), and output the index. The calculation result by the calculating portion (1002) in the embodiment refers to “the total amount of the intensity of each frequency within a unit time period” of the measurement wave, and this represents the “magnitude of the vibration movement of the diaphragm within the unit time period”. Therefore, the calculation result by the calculating portion calculated when laughter is detected can be considered to represent the “magnitude of the laughter within the unit time period”. The “unit time period” is an arbitrary time period that can be set by a user. For example, when the laughter measurement apparatus includes the comparing portion that is described in the second embodiment, the “unit time period” may be determined to be equal to the one unit time period during which the comparing portion executes the comparison (examples: “500 msec” or “6 sec”). Otherwise, the total measurement time period (examples: “30 minutes” or “one hour”) may be set as the unit time period.

The calculation result by the calculating portion (1002) is a numerical value without any unit. Therefore, even when this numerical value is supplied as it is to the subject as the magnitude of his/her laughter, it is difficult for the subject to guess how large the numerical value is. Therefore, the laughter amount output portion (1007) is adapted to calculate and convert the calculation result of the calculating portion (1002) into an index that is understandable for the subject as the magnitude of the laughter, and output the index. For example, a specific calculating means may retain the “total amount of the intensity of each frequency within the unit time period” calculated based on ordinary laughter of a human in advance as a reference value. The specific calculating means may calculate relative magnitude of the “total amount of the intensity of each frequency within the unit time period of the subject” setting the reference value to be “100”. When the calculation result is output, the calculation result may not only be provided as numerical values but also be provided with some unit attached thereto to make the numerical values accessible. For example, the laughter reaction may also be represented as “195 aH” with the unit “aH (aha)”. The calculation result of the laughter amount output portion (1007) is output every unit time period. Therefore, when the unit time period is set to be equal to one unit time period for the comparing portion to execute the comparison, the comparison result by the comparing portion and the calculation result by the laughter amount output portion are output every unit time period. In this case, the results can be output as “laughter-200 aH”, etc., by combining the results with each other.

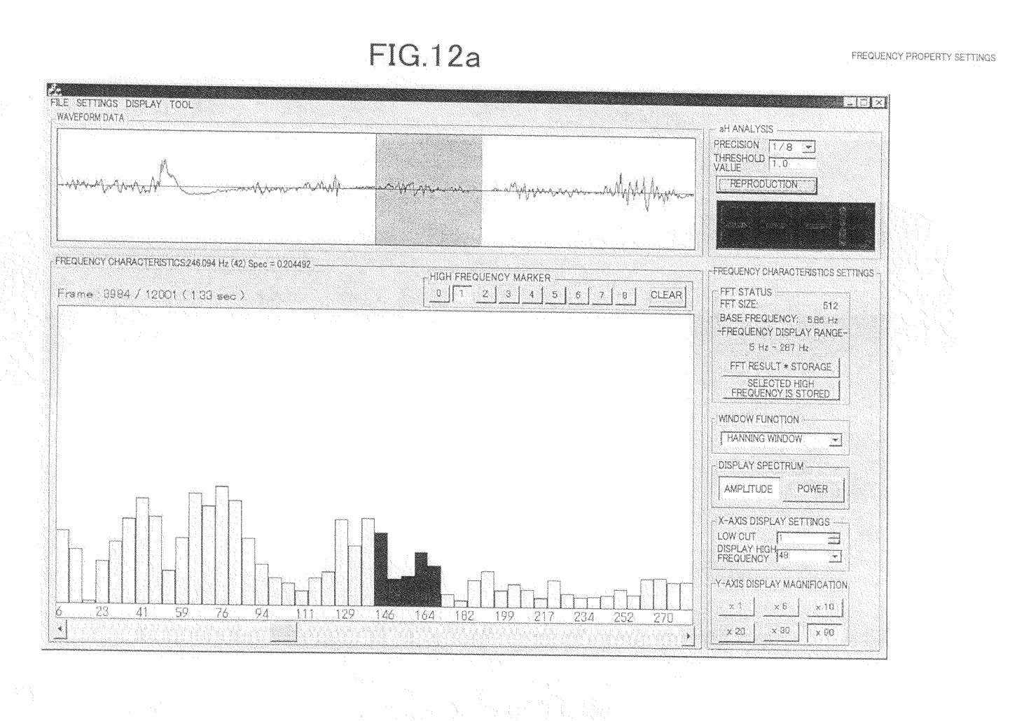

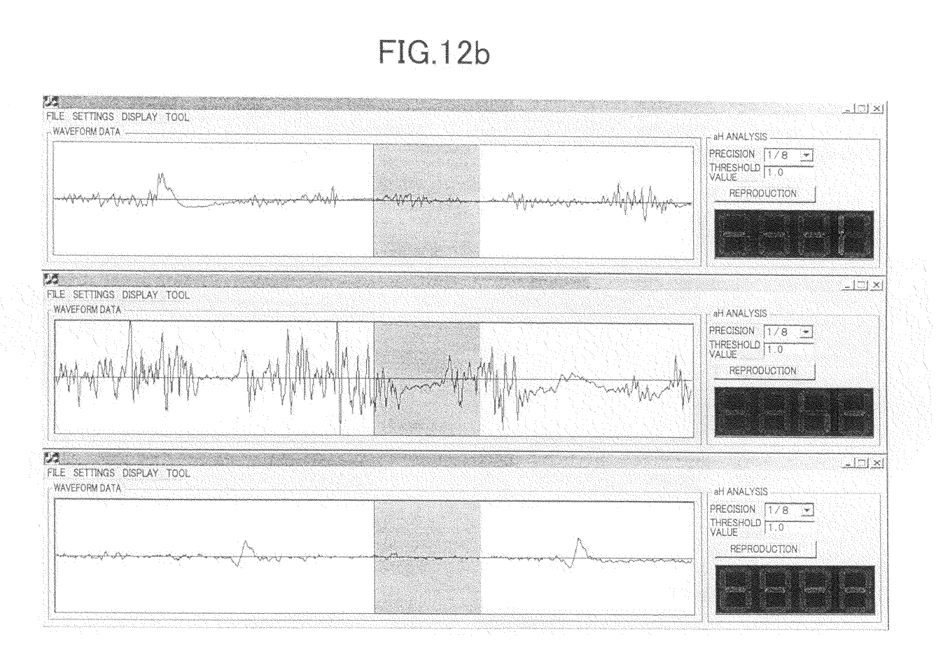

FIG. 12(a) shows an example of a screen that outputs the “index that indicates the magnitude of laughter” that the calculating portion (1002) frequency-analyzed the potential measurement wave measured by the potential measuring portion (1001) and the laughter amount output portion (1107) calculated. In FIG. 12(a), “waveform data” in the upper-left portion is the potential measurement wave measured by the potential measuring portion (the axis of abscissa: time, the axis of ordinate: voltage). “Frequency characteristics” thereunder is the result shown as a graph (the axis of abscissa: frequency, the axis of ordinate: intensity) obtained by frequency-analyzing data in a portion identified by being colored of the “waveform data”. A digitally displayed portion in “aH analysis” in the upper-right portion of FIG. 12(a) is a portion that outputs the index that indicates the magnitude of laughter.

FIG. 12(b) shows another exemplary output. FIG. 12(b) shows an exemplary output obtained when the amount of laughter of each of three subjects simultaneously is measured. Data represented in each of the “waveform data” in the left portion of FIG. 12 (b) and the “aH analysis” in the right portion thereof are the same as those described in FIG. 12(a).

The exemplary outputs are examples and the output is not limited to these.

The processing of the “laughter amount output portion” (1007) may be executed after removing noises such as a heart beat component. More specifically, the heart beat component may be removed by removing the data at frequencies of 20 to 50 Hz or lower. As also shown in FIG. 7, a strong reaction is seen between 50 and 500 Hz in the potential wave measured during the laughter reaction. Therefore, the laughter amount may be measured for a frequency band (examples: “50 to 140 Hz” or “140 to 500 Hz”) that is arbitrarily determined in the above range.

### <Effects of Third Embodiment>

According to the laughter measurement method and the measurement apparatus of the embodiment, the magnitude of laughter detected from a subject can be converted into a form that is understandable for the subject and be provided to the subject.

As a result, the subject can easily grasp the measured magnitude of the subject's own laughter, and can easily compare the magnitude with the subject's own past data and data of another person and, thereby, can grasp the difference among them.

### <<Fourth Embodiment>>

### <Overview of Fourth Embodiment>

A laughter measurement apparatus of a fourth embodiment is based on that of the first, the second, or the third embodiment, and is characterized in that a potential measuring portion that measures a potential from a subject is installed in a housing and the housing has an electrode to be attached tightly to the subject on its side.

### <Functional Configuration of Fourth Embodiment>

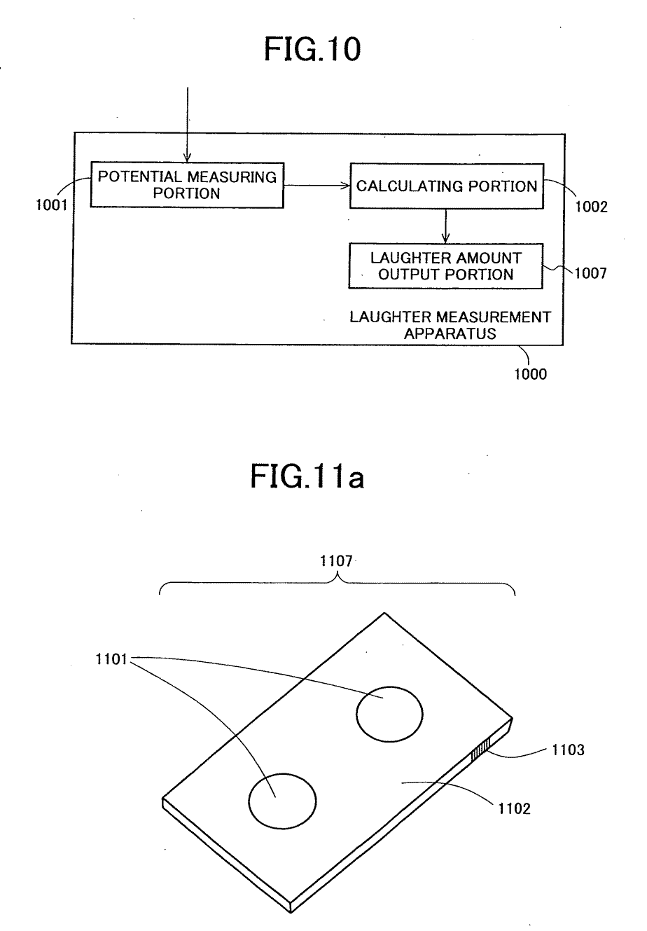

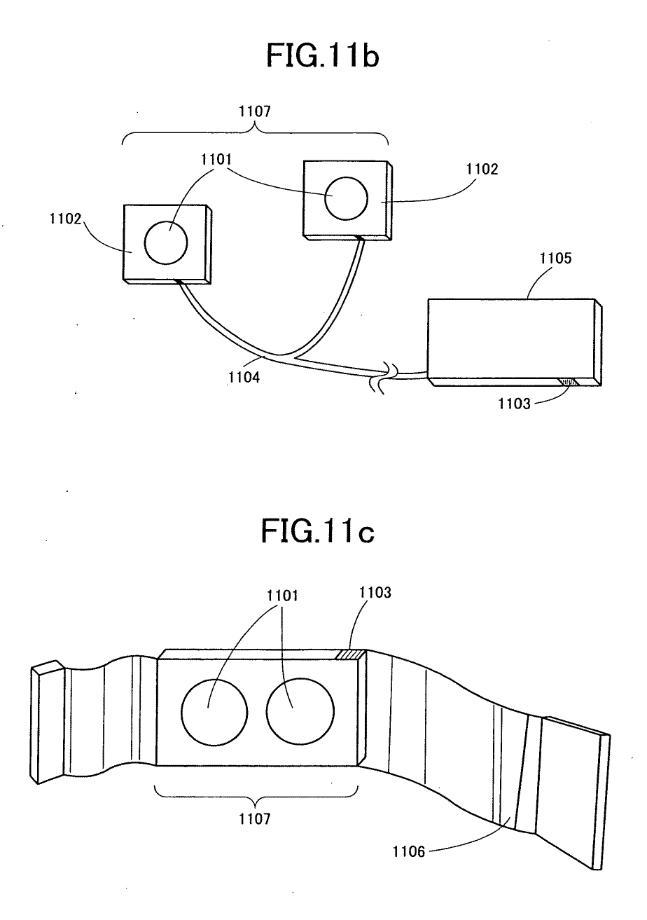

The potential measuring portion of the embodiment is installed in the housing and has electrodes to measure the potential adhering to a skin of a human on one side. FIG. 11 depicts examples of the housing of the embodiment. As to a housing (1107) depicted in FIG. 11(a), a portion (1102) that is the rest of the side provided with the electrodes (1101) may be applied with treatment for the portion (1102) to adhere to a skin of a human. For example, the portion (1102) may be applied with treatment using an adhesive generally used to fix the portion (1102) onto a skin. By applying treatment in this manner, the housing (1107) that installs the potential measuring portion can easily be adhered onto a skin of a human. A memory to store the measured data may be provided in the housing. In this case, the portion (1102) is provided with an interface (1103) to communicate with an external device and, after storing the measured data in the memory, the data may also be transmitted to the external device that is provided with an integrating operation portion, etc.

As depicted in FIG. 11(b), the housing (1107) of the embodiment may be divided into two portions, and may be connected to a small apparatus (1105) that has the memory to store the measured data with a cord (1104), etc. By configuring as above, it is possible to downsize the housing and to make measurement easily. The portion (1102) that is the rest of the face provided with an electrode (1101) of a divided housing may also be applied with the same treatment as above. The small apparatus (1105) that stores the measured data may be provided with the interface (1103) to communicate with the external device.

As depicted in FIG. 11(c), the housing (1107) of the embodiment may also be provided with a belt (1106), etc. In this case, the housing having the potential measuring portion can be fixed to a subject with the belt (1106) and, therefore, the portion (1102) that is the rest of the face provided with the electrodes (1101) does not need to be applied with any treatment using an adhesive, etc., as above. The housing (1107) may also be provided with a memory to store the measured data and the interface (1103) to communicate with an external device.

### <Effects of Fourth Embodiment>

The laughter measurement apparatus of the embodiment is a small and highly convenient apparatus. As a result, the apparatus can be easily used in not only research institutions, etc., but also ordinary homes, etc. Because the small apparatus can measure and can have the data stored in the apparatus, it is possible to measure people simultaneously as subjects in a site of a comical performance event, etc.

<<Advantage of Detecting Laughter (Vibration Movement of Diaphragm) Using Skin Surface Potential of Surface of Bone Tissue Coupled with Tendon of Starting Portion of Diaphragm or Starting Portion of Diaphragm (such as “Xiphoid Process”, “Vicinity on Seventh Right Rib”) of Subject“>>

The data will be described that indicates advantage of detecting laughter (the vibration movement of the diaphragm) using the skin surface potential of the surface of the bone tissue coupled with the tendon of the starting portion of the diaphragm or the starting portion of the diaphragm of a subject. An example of the “xiphoid process” will be described as “the surface of the bone tissue coupled with the tendon of the starting portion of the diaphragm or the starting portion of the diaphragm of a subject”. However, the same description is applied to the other portions (such as “the vicinity on the seventh right rib”).

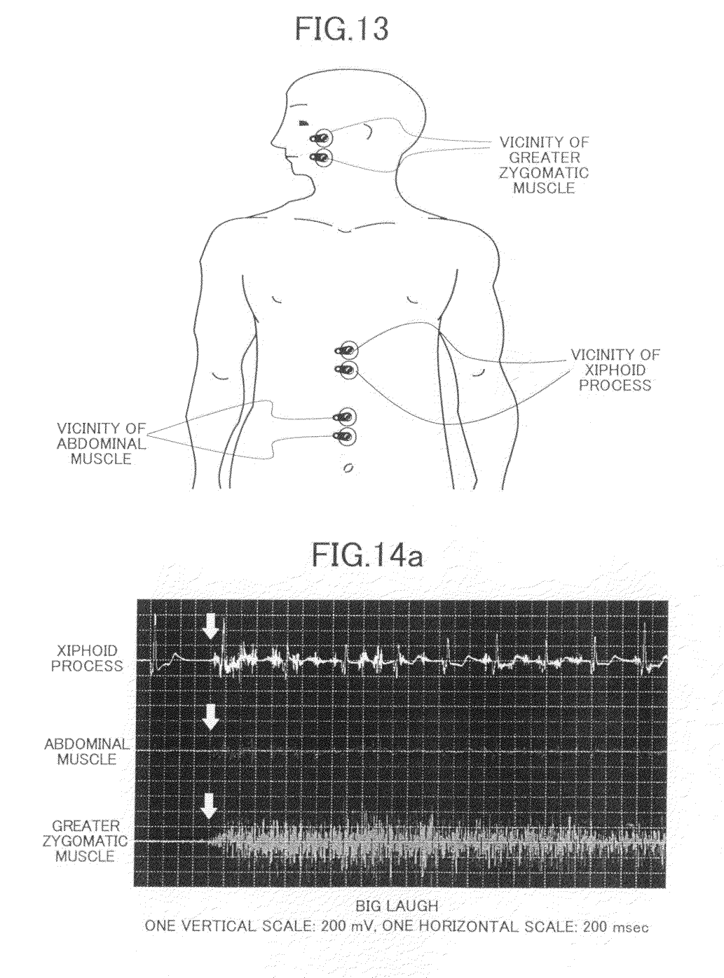

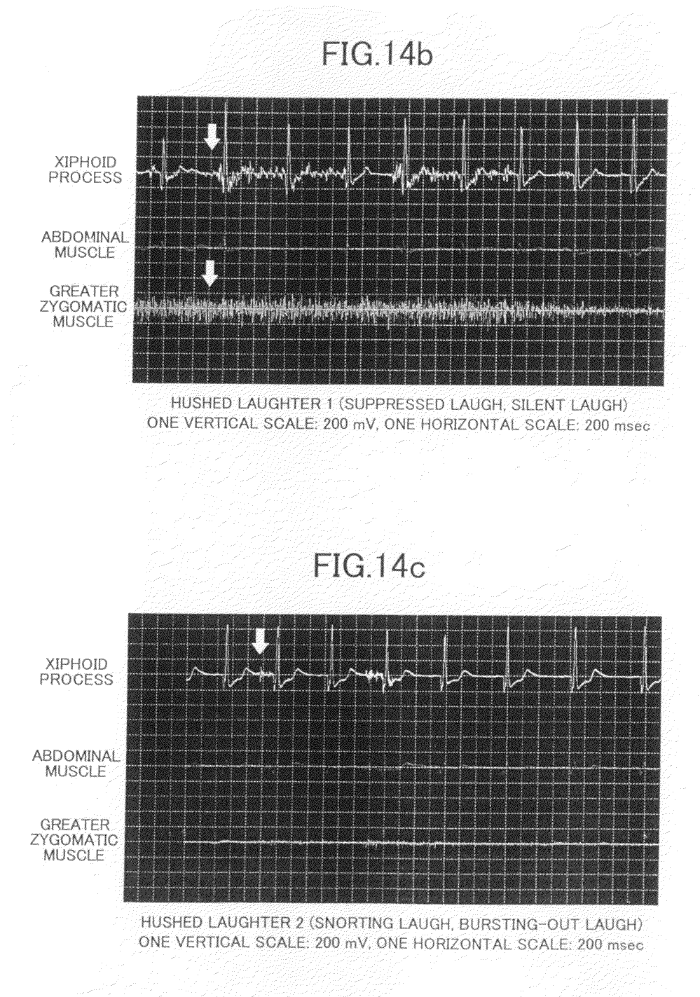

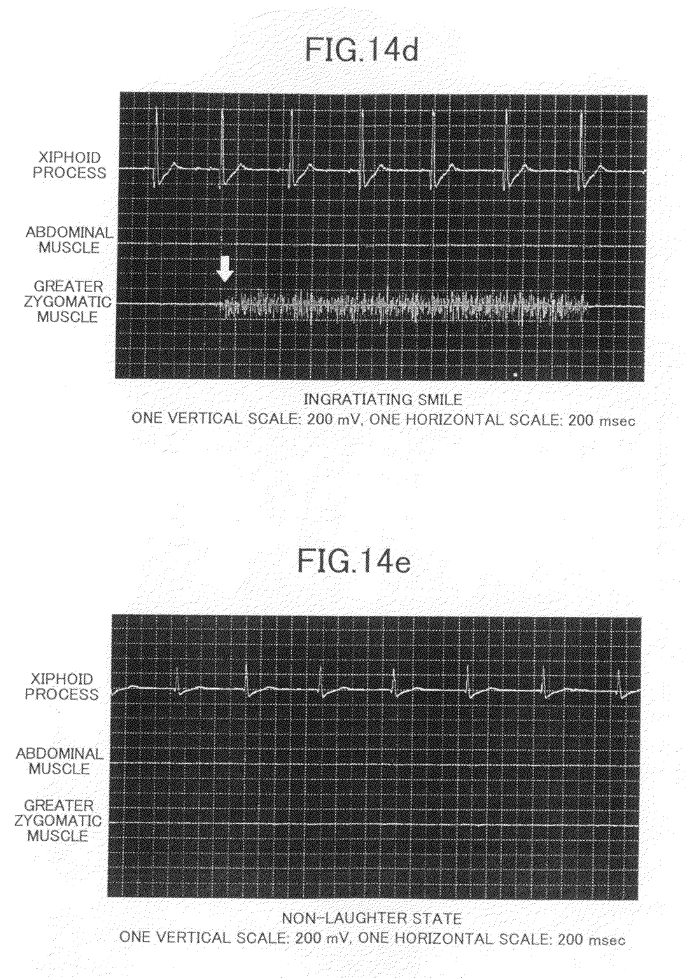

FIGS. 14(a) to (e) show examples of potential measurement waves obtained when skin surface potentials in the vicinities of three points that are “xiphoid process”, “abdominal muscle”, and “greater zygomatic muscle” are measured (hereinafter, “three-point measurement”) (with the axis of abscissa representing time and the axis of ordinate representing voltage). As shown in FIG. 13, FIGS. 14(a) to (e) show the potential measurement waves obtained when the skin surface potentials in the vicinity of the xiphoid process, the vicinity of the abdominal muscle, and the vicinity of the greater zygomatic muscle are simultaneously measured. The three measurement waves are potential measurement waves in downward order of the vicinity of xiphoid process, the vicinity of the abdominal muscle, and the vicinity of the greater zygomatic muscle. In this manner, the potential measurement waves are compared and verified of the three points obtained during “big laughter”, “hushed laughter 1 (a suppressed laugh, a silent laugh)”, “hushed laughter 2 (a snorting laugh, bursting-out laughter)”, “hushed laughter 3 (a laughter without any voice) ”, “a forced laugh”, “an ingratiating smile”, “a vacuous smile (only a voice)”, and “a non-laughter state (ordinary state)” of a subject. For reference, a portion of each of the potential measurement waves is shown in FIGS. 14(a) to (e).

FIG. 14(a) shows the potential measurement waves obtained during the “big laughter” of the subject. FIG. 14(b) shows the potential measurement waves obtained during the “hushed laughter 1 (the suppressed laugh, the silent laugh)” of the subject. FIG. 14(c) shows the potential measurement waves obtained during the “hushed laughter 2 (the snorting laugh, the bursting-out laughter)” of the subject. FIG. 14(d) shows the potential measurement waves obtained during the “ingratiating smile” of the subject. FIG. 14(e) shows the potential measurement waves obtained during the non-laughter state (ordinary state) ” of the subject. The waveform of the location having an observable reaction is indicated by an arrow.

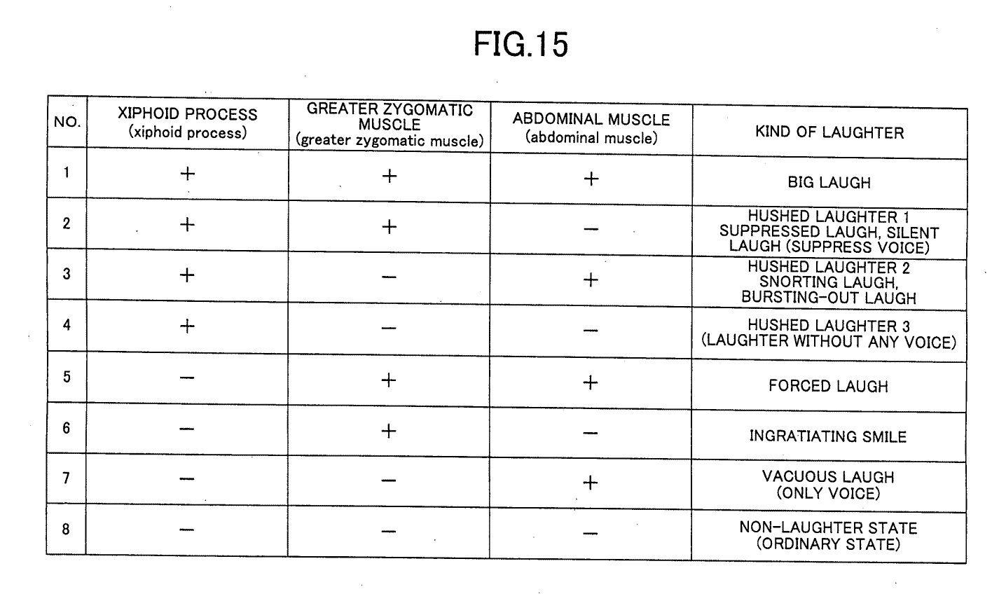

FIG. 15 shows the result of the three-point measurement. The symbol, “+”, in a table indicates that a reaction is detected that is caused by a factor other than heart beats such as a significant increase of the amplitude of a potential measurement wave. The symbol, “−”, in the table indicates that any reaction is not detected at all that is caused by a factor other than heart beats in a potential measurement wave. Determination of whether a potential measurement wave is caused by heart beats is made by measuring in advance a potential measurement wave caused by heart beats (non-laughter state (ordinary state)) (FIG. 14(e)) and comparing the potential measurement wave with the data.

As shown in FIG. 15, the potential measurement waves in the vicinity of the xiphoid process indicates “+” for the “big laughter”, the “hushed laughter 1 (the suppressed laugh, the silent laugh)”, the “hushed laughter 2 (the snorting laugh, the bursting-out laughter)”, and the “hushed laughter (a laughter without any voice)” and indicates “−” for the “forced laugh”, the “ingratiating smile”, the “vacuous laugh (only voice)”, and “non-laughter state (ordinary state)”. From the result, the potential measurement wave in the vicinity of the xiphoid process does not react for so-called “false laughter” such as the “forced laughter”, the “ingratiating smile”, and the “vacuous laugh (only voice)”, and only reacts for “real laughter” such as the “big laugh”, the “hushed laughter 1 (the suppressed laugh, the silent laugh)”, “hushed laughter 2 (the snorting laugh, the bursting-out laughter)”, and the “hushed laughter (the laughter without any voice)”. Only the “real laughter” can be detected using the potential measurement wave in the vicinity of the xiphoid process.

On the other hand, it can be seen that the potential measurement waves in the vicinity of the abdominal muscle and the vicinity of the greater zygomatic muscle do not react for the “hushed laughter 1, 2, and 3” and react during the “forced laugh”, the “ingratiating smile”, the “vacuous laugh”, etc. Only the “real laughter” can not accurately be detected using the potential measurement waves in the vicinity of the abdominal muscle and the vicinity of the greater zygomatic muscle.

1. A laughter measurement method comprising:
a potential measuring step for measuring a skin surface potential of a surface of a bone tissue that is coupled with a tendon of a starting portion of a diaphragm or the starting portion of the diaphragm of a subject over time; and
a calculating step for calculating a variation over time of an intensity of each frequency of a measurement wave that represents the variation over time of the potential measured.

2. The laughter measurement method as defined in claim 1, wherein
the surface of the bone tissue that is coupled with the tendon of the starting portion of the diaphragm or the starting portion of the diaphragm of the subject is vicinity of xiphoid process.

3. The laughter measurement method as defined in claim 1, wherein
the surface of the bone tissue that is coupled with the tendon of the starting portion of the diaphragm or the starting portion of the diaphragm of the subject is vicinity of a location on a seventh rib that is positioned about 10 cm away and substantially horizontally rightward from the xiphoid process.

4. The laughter measurement method as defined in claim 1, further comprising:
a preparing step for preparing a reference pattern formed by mapping an intensity pattern of each frequency using one axis as a frequency axis and another axis as a time axis, as a reference pattern of a measurement wave measured during laughter;
a mapping step for sampling intensity data of each frequency in the measurement wave of the subject calculated at the calculating step and mapping the intensity data sampled of the subject using one axis as a frequency axis and another axis as a time axis;
a comparing step for comparing the intensity pattern of the subject mapped at the mapping step with the reference pattern prepared in advance at the preparing step; and
a comparison result output step for outputting information that identifies whether laughter is detected according to a result of the comparison at the comparing step.

5. The laughter measurement method as defined in claim 1, further comprising
a laughter amount output step for calculating and outputting an index that indicates magnitude of laughter according to a result of the calculation at the calculating step.

6. A laughter measurement apparatus comprising:
a potential measuring portion that measures a skin surface potential of a surface of a bone tissue that is coupled with a tendon of a starting portion of a diaphragm or the starting portion of the diaphragm of a subject over time; and
a calculating portion that calculates a variation over time of an intensity of each frequency of a measurement wave that represent the variation over time of the potential measured.

7. The laughter measurement apparatus as defined in claim 6, wherein
the potential measuring portion comprises a xiphoid process vicinity measuring means that measures a skin surface potential in the vicinity of the xiphoid process over time as the skin surface potential of the surface of the bone tissue that is coupled with the tendon of the starting portion of the diaphragm or the starting portion of the diaphragm of the subject.

8. The laughter measurement apparatus as defined in claim 6, wherein
the potential measuring portion comprises a seventh rib vicinity measuring means that measures a skin surface potential in the vicinity of a location on a seventh rib that is positioned about 10 cm away and substantially horizontally rightward from the xiphoid process over time as the skin surface potential of the surface of the bone tissue that is coupled with the tendon of the starting portion of the diaphragm or the starting portion of the diaphragm of the subject.

9. The laughter measurement apparatus as defined in claim 6, wherein
the potential measuring portion is installed in a housing, and wherein
an electrode to measure a potential by contacting with a skin of a human is provided on one side of the housing.

10. The laughter measurement apparatus as defined in claim 6, further comprising:
a reference pattern retaining portion that retains a reference pattern formed by mapping an intensity pattern of each frequency using one axis as a frequency axis and another axis as a time axis, as a reference pattern of a measurement wave measured during laughter;
a mapping portion that samples intensity data of each frequency in the measurement wave of the subject calculated by the calculating portion, the mapping portion mapping the intensity data sampled of the subject using one axis as a frequency axis and another axis as a time axis;
a comparing portion that compares the intensity pattern of the subject mapped by the mapping portion with the reference pattern retained in the reference pattern retaining portion; and
a comparison result outputting portion that outputs information that identifies whether laughter is detected according to a result of the comparison by the comparing portion.

11. The laughter measurement apparatus as defined in claim 6, further comprising
a laughter amount outputting portion that calculates and outputs an index that indicates magnitude of laughter according to a result of the calculation by the calculating portion.

12. The laughter measurement method as defined in claim 2, further comprising
a laughter amount output step for calculating and outputting an index that indicates magnitude of laughter according to a result of the calculation at the calculating step.

13. The laughter measurement method as defined in claim 3, further comprising
a laughter amount output step for calculating and outputting an index that indicates magnitude of laughter according to a result of the calculation at the calculating step.

14. The laughter measurement method as defined in claim 4, further comprising
a laughter amount output step for calculating and outputting an index that indicates magnitude of laughter according to a result of the calculation at the calculating step.

15. The laughter measurement apparatus as defined in claim 7, further comprising
a laughter amount outputting portion that calculates and outputs an index that indicates magnitude of laughter according to a result of the calculation by the calculating portion.

16. The laughter measurement apparatus as defined in claim 8, further comprising
a laughter amount outputting portion that calculates and outputs an index that indicates magnitude of laughter according to a result of the calculation by the calculating portion.

17. The laughter measurement apparatus as defined in claim 9, further comprising
a laughter amount outputting portion that calculates and outputs an index that indicates magnitude of laughter according to a result of the calculation by the calculating portion.

18. The laughter measurement apparatus as defined in claim 10, further comprising
a laughter amount outputting portion that calculates and outputs an index that indicates magnitude of laughter according to a result of the calculation by the calculating portion.

19. The laughter measurement method as defined in claim 2, further comprising:
a preparing step for preparing a reference pattern formed by mapping an intensity pattern of each frequency using one axis as a frequency axis and another axis as a time axis, as a reference pattern of a measurement wave measured during laughter;
a mapping step for sampling intensity data of each frequency in the measurement wave of the subject calculated at the calculating step and mapping the intensity data sampled of the subject using one axis as a frequency axis and another axis as a time axis;
a comparing step for comparing the intensity pattern of the subject mapped at the mapping step with the reference pattern prepared in advance at the preparing step; and
a comparison result output step for outputting information that identifies whether laughter is detected according to a result of the comparison at the comparing step.

20. The laughter measurement method as defined in claim 3, further comprising:
a preparing step for preparing a reference pattern formed by mapping an intensity pattern of each frequency using one axis as a frequency axis and another axis as a time axis, as a reference pattern of a measurement wave measured during laughter;
a mapping step for sampling intensity data of each frequency in the measurement wave of the subject calculated at the calculating step and mapping the intensity data sampled of the subject using one axis as a frequency axis and another axis as a time axis;
a comparing step for comparing the intensity pattern of the subject mapped at the mapping step with the reference pattern prepared in advance at the preparing step; and
a comparison result output step for outputting information that identifies whether laughter is detected according to a result of the comparison at the comparing step.
